(19) United States
(12) Patent Application Publication
(10) Pub. No.: US 2024/0226196 A9
(43) Pub. Date: Jul. 11, 2024

(54) Composition and Use Thereof

(71) Applicant: Axisbiotix Limited, Macclesfield Cheshire (GB)

(72) Inventor: Catherine O'Neill, Macclesfield Cheshire (GB)

(21) Appl. No.: 18/546,321

(22) PCT Filed: Feb. 17, 2022
(86) PCT No.: PCT/GB2022/050428

§ 371 Date: Aug. 14, 2023

(30) Foreign Application Priority Data
Feb. 17, 2021 (GB) 2102228.0
May 20, 2021 (GB) 2107254.1

(51) Int. Cl. A61K 35/745; A61K 9/00; A61K 35/744; A61K 35/747; A61P 17/00; A61P 17/06

(52) U.S. Cl. CPC A61K 35/745; A61K 9/009; A61K 9/0095; A61K 35/744; A61K 35/747; A61P 17/00; A61P 17/06; A61K 2035/115

(57) ABSTRACT

The present invention relates to a composition for use in the treatment and/or prevention of a dry and/or sensitive skin condition in a subject. The composition comprises at least two bacterial species which decrease the levels of interleukin-17 and/or interleukin-23 in the subject, and/or increase the levels of interleukin-10 in the subject and/or improve gut barrier function in the subject. The at least two bacterial species may comprise Bifidobacterium animalis, Lactobacillus brevis, Lactobacillus reuteri and/or Lactococcus lactis.

### COMPOSITION AND USE THEREOF

### Field of the Invention

The present invention relates to a composition and the use of said composition in the treatment or prevention of dry or sensitive skin conditions, for example psoriasis.

### Background to the Invention

Humans live in constant contact with a multitude of microorganisms. The gut is by far the most heavily colonised and the gut microbiota has been shown to play multiple roles in normal physiology including nutrient sequestration and the development of normal immune responses. In addition, the gut microbiota has increasingly been shown to play a role in disease and the treatment and management of disease.

Amongst the normal gut microbiota are the so-called probiotic bacteria. Probiotics have been defined as live microorganisms which, when administered in adequate amounts, confer a health benefit to the host. The concept that probiotics are beneficial to gut health has been investigated at length and studies have demonstrated that probiotics potentially improve gut function through several mechanisms including increasing epithelial barrier function and modulating the immune response. There is also evidence that probiotics can prevent colonisation of the gut by pathogens, for example by downregulating virulence factors and/or inhibiting pathogen adherence to the epithelium.

Ingestion of probiotic bacteria has been shown to prevent or treat gastrointestinal disorders such as antibiotic-associated diarrhoea and inflammatory bowel disease. However, in general, there is comparatively little information available regarding the molecular mechanisms mediating the observed effects of probiotics.

Since probiotics may have positive impacts on the gut, their potential effects on other systems, such as the mouth and the urogenital tract have also begun to be investigated and the topical application of probiotics to the skin has been investigated in a limited number of studies. There is compelling evidence suggesting that gastrointestinal (GI) disorders are linked to several inflammatory skin diseases such as rosacea, psoriasis and atopic dermatitis. Psoriatic sufferers have an increased risk of developing gut inflammation and between 7-11% of inflammatory bowel disease patients are also diagnosed with psoriasis. Studies have also shown that individuals with psoriasis and atopic dermatitis have a less diverse gut microbiome which may be linked to a reduction in the ability of the gut to regulate immune responses and a localised inflammatory response. Changes in the gut microbiome are also linked to immune system reactivity and a disruption in skin homeostasis and inflammatory skin diseases. Additionally, other mechanisms involved in the gut-skin axis have been demonstrated, which include the gut microbiotas capacity to synthesise molecules that can access the bloodstream and subsequently accumulate in the skin and the effect of the gut microbiota on mood, which can affect skin condition. Although these mechanisms are not fully understood yet, there is undeniable evidence demonstrating the relationship between the gut microbiome and skin health which involves a connection between nervous, immune and endocrine systems as well as environmental factors.

Psoriasis is a complex chronic inflammatory condition which manifests as scaling, erythema and disfiguring dry skin plaques that are painful and itchy. These lesions are normally found on the limbs and scalp. Psoriasis has a profound psychological and social effect and can induce isolation, affect employment and lead to anxiety and depression. It is also associated with co-morbidities such as inflammatory bowel disease, cardiovascular disease and diabetes. The severity of psoriasis varies from 1-2% skin plaques (mild) to nearly entire body coverage (severe) and can be classified into the following types: plaque, guttate, inverse, pustular and erythrodermic. Plaque psoriasis is the most common form of psoriasis, accounting for around 85-90% of cases. This type of psoriasis typically presents as red-coloured patches, with white or silvery scales on top which are most commonly seen on the elbows, knees and scalp. With a prevalence of 2-4% in the UK, the economic impact of psoriasis exceeds £1 bn p.a.

Although the processes associated with the development of psoriasis are not fully understood, psoriasis has an immune component and is now often considered to be an autoimmune condition, in which the immune system attacks healthy skin cells. In addition, as mentioned above, psoriatic sufferers have a less diverse gut microbiome and a ‘leaky gut’ has been observed in individuals with psoriasis. In mouse models of psoriasis, modulation of the gut microbiome impacts upon the skin. Although it has not been determined how the microbiome is able to exert an effect on the skin, it is thought this may be related to the modulatory effect of probiotics on systemic immunity.

There is no cure for psoriasis although a number of treatment options are available. Care for patients falls under the guidance of a dermatologist with many individuals also purchasing supportive products in an attempt to reduce the severity and coverage of lesions. Therapeutics are effective; however, few patients have well controlled disease and treatment dissatisfaction with medications that have considerable side effect profiles is pronounced.

Due to the suspected immune origin of psoriasis, many treatments have focused on the use of immunosuppressants, both topical and systemic. In mild forms of the condition, topical treatments such as vitamin D or corticosteroids are often employed. Phototherapy and systemic treatments are utilised in more severe cases. Patients with psoriasis often exhibit elevated levels of inflammatory markers such as interleukins (particularly interleukin-17 (IL-17) and interleukin-23 (IL-23)) and more recently, there has been focus on developing treatments which modulate the levels of such inflammatory markers or impact the molecular pathways associated with the immune pathway.

Atopic Dermatitis (AD), also known as atopic eczema, is a chronic inflammatory skin condition. AD is the most common form of eczema and currently affects approximately 20% of the paediatric population in the western world. AD causes the skin to become itchy, red, dry and cracked. Scratching worsens symptoms and affected individuals have an increased risk of skin infections. If the skin barrier is breached irritants such as soaps and detergents can dry the skin and further deteriorate the weakened skin barrier. Food, microbes and other allergens can also penetrate the upper layers of the skin potentially causing allergic reactions. Some people may only have small patches of dry skin, but others may experience widespread red, inflamed skin all over the body.

Treatments used to control symptoms and manage AD include self-care techniques, such as reducing scratching and avoiding triggers, emollients (moisturising treatments) and topical corticosteroids, used to reduce swelling, redness and itching during flare-ups.

Topical corticosteroids can cause a mild stinging sensation upon application and in rare cases can result in thinning of the skin, skin thickening, stretch marks, changes in skin colour, acne and increased hair growth. Less common but more severe side effects of steroids include glaucoma, cataracts, tiny pink bumps on the skin, red pus-filled hair follicles, adrenal suppression and topical steroid addiction/withdrawal. For particularly severe flare-ups corticosteroid tablets may be prescribed but courses of treatment longer than about 7 days are generally avoided because of the risk of potentially serious side effects.

Alternative non-steroidal AD treatments include topical calcineurin inhibitors, such as pimecrolimus and tacrolimus, that suppress the immune system, phototherapy, bandages or wet wraps, immunosuppressant tablets and alitretinoin to treat severe eczema affecting the hands in adults. However, there have been concerns that calcineurin inhibitors may increase the risk of skin cancer or lymphoma.

Ichthyosis is a condition that causes widespread and persistent thick, dry, scaly skin. There are more than twenty different types of ichthyosis which range in the severity of symptoms and appearance. In the most common type of ichthyosis, ichthyosis vulgaris, the symptoms are relatively mild. However, the most severe types of ichthyosis can be life threatening.

The treatment of ichthyosis typically includes the topical use of emollients and creams and exfoliation to remove the scales. In more severe cases, the oral use of retinoids may be prescribed, to reduce the production of skin cells. However, treatments options are limited.

It is an object of the present invention to obviate or mitigate one or more of the abovementioned problems and/or provide improved treatments for dry and/or sensitive skin conditions such as psoriasis, atopic dermatitis and ichthyosis.

### Summary of the Invention

The present invention relates to a composition and the use of a composition in the treatment or prevention of dry and/or sensitive skin conditions. The present invention is based, in part, on studies by the inventors in which they have shown that certain probiotic bacterial species are able to decrease the expression of interleukin-17 (IL-17) and interleukin-23 (IL-23), increase the expression of interleukin-10 (IL-10) and improve gut barrier function. The present inventors have shown that the oral use of such probiotic bacterial species, particularly a blended population of such probiotic bacterial species, results in improved skin health.

In a first aspect of the present invention there is provided a composition for use in the treatment and/or prevention of a dry and/or sensitive skin condition in a subject, wherein the composition comprises at least two bacterial species which decrease the levels of interleukin-17 and/or interleukin-23 in the subject, and/or increase the levels of interleukin-10 in the subject and/or improve gut barrier function in the subject.

In a second aspect of the invention, there is provided a composition comprising at least two bacterial species which decrease the levels of interleukin-17 and/or interleukin-23, and/or increase the levels of interleukin-10 and/or improve gut barrier function.

The bacterial species may be probiotic bacterial species. The composition of the present invention can therefore be described as a probiotic composition as it may comprise at least two probiotic bacterial species. Probiotic bacteria are defined by the World Health Organisation as live microorganisms which when administered in adequate amounts confer a health benefit on the host. Therefore, as will be appreciated by the skilled person, the “probiotic composition” of the present invention is a composition comprising at least two probiotic bacterial species, which, when applied to a subject produce a beneficial effect in the subject.

The present inventors have found that by managing the inflammatory response in individuals with dry or sensitive skin conditions, these conditions can be treated. Many such conditions, for example psoriasis, are associated with an uncontrolled or overactive inflammatory response and by reducing this inflammatory response to more normal levels (i.e. the levels seen in subjects not affected by such conditions), the conditions can be managed. By utilising a blend of bacterial species (rather than a single strain approach) the present inventors have developed a composition which can capitalize on the benefits of using blended populations of probiotics that target different physiological aspects of skin disease.

The phrase “decrease the levels of interleukin(s)” as used herein means that the interleukin levels in the subject are decreased as compared to the levels before treatment with the bacterial species. By decreasing the level of interleukins (e.g. IL-17, IL-23) in subjects who have a dry or sensitive skin condition, for example psoriasis, the levels of these interleukins are reduced to the level of interleukins observed in a subject without said dry or sensitive skin condition.

The phrase “increase the levels of interleukin(s)” as used herein means that the interleukin levels in the subject are increased as compared to the levels before treatment with the bacterial species. By increasing the level of interleukins (e.g. IL-10) in subjects who have a dry or sensitive skin condition, for example psoriasis, the level of these interleukins may be increased to at least the level of interleukins observed in a subject without said dry or sensitive skin condition.

As the skilled person will appreciate, there are various ways in which the levels of interleukins can be measured, including, for example by testing the concentration of said interleukins in the blood using an enzyme linked immunosorbent assay (ELISA).

As discussed above, dry and sensitive skin conditions, for example psoriasis, have been found to be associated with an altered microbiome and a leaky gut. The phrase “improve gut barrier function” as used herein means that the function of the gut in preventing translocation of pathogens and other noxious molecules across the gut is improved as compared to the function before treatment with the bacterial species. The gut function is improved so that it is more comparable to that of a subject without said dry or sensitive skin condition. The skilled person will appreciate that gut function can be tested, for example, by measuring the ratio of mannitol:lactulose in the urine following oral ingestion of such sugars.

As will be appreciated by the skilled person, when referring to the bacterial species in the composition which decrease interleukin levels, and/or increase interleukin levels and/or improve gut function, we refer to the interleukin levels and/or gut function in a subject to whom the composition has been administered.

In embodiments, the bacterial species may be selected from the group consisting of Lactobacillus sp., Bifidobacterium sp., Lactococcus sp., Streptococcus sp. and/or combinations thereof. In embodiments, the bacterial species may be selected from the group consisting of Lactobacillus sp., Bifidobacterium sp., Lactococcus sp. and/or combinations thereof.

The bacterial species of the present invention may be selected from Bifidobacterium animalis, Lactobacillus brevis, Lactobacillus reuteri, Lactococcus/lactis/Lactococcus cremoris, Lactobacillus acidophilus, Lactobacillus casei, Bifidobacterium infantis, Lactobacillus plantarum, Lactobacillus pentosus, Lactobacillus paracasei, Lactobacillus acidophilus and Bifidobacterium breve, for example.

In embodiments of the present invention, the composition comprises at least two of the following bacterial species: Bifidobacterium animalis, Lactobacillus brevis, Lactobacillus reuteri and Lactococcuslactis.

Lactococcuslactis can also be known as Lactococcus cremoris. Therefore, in embodiments, the composition comprises at least two of the following bacterial species: Bifidobacterium animalis, Lactobacillus brevis, Lactobacillus reuteri and Lactococcus cremoris.

The present invention therefore provides a composition for use in the treatment and/or prevention of a dry and/or sensitive skin condition in a subject, wherein the composition comprises at least two of the following bacterial species: Bifidobacterium animalis, Lactobacillus brevis, Lactobacillus reuteri and Lactococcus/lactis/Lactococcus cremoris.

The present invention also provides a composition comprising at least two of the following bacterial species: Bifidobacterium animalis, Lactobacillus brevis, Lactobacillus reuteri and Lactococcus/lactis/Lactococcus cremoris.

Bifidobacterium animalis (B. animalis) is a gram-positive, anaerobic, rod-shaped bacterium which is found in the gastrointestinal tract of most mammals, including humans. The scientific classification of B. animalis is: Kingdom: Bacteria; Division:

Firmicutes; Class: Actinobacteria; Order: Bifidobacteria; Family: Bifidobacteriaceae; Genus: Bifidobacterium; Species: Bifidobacterium animalis. B. animalis is commonly used as a probiotic.

Lactobacillus brevis (L. brevis) is a gram-positive, rod-shaped, lactic acid bacterium commonly found in milk products, for example. The scientific classification of L. brevis is: Kingdom: Bacteria; Division: Firmicutes; Class: Bacilli; Order: Lactobacillales; Family: Lactobacilliaceae; Genus: Lactobacillus; Species: Lactobacillus brevis.

Lactobacillus reuteri (L. reuteri) is a gram-positive, rod-shaped, anaerobic bacterium which is found in the gastrointestinal tract of most mammals, including humans. The scientific classification of L. reuteri is: Kingdom: Bacteria; Division: Firmicutes; Class: Bacilli; Order: Lactobacillales; Family: Lactobacilliaceae; Genus: Lactobacillus; Species: Lactobacillus reuteri.

Lactococcus lactis (L. lactis) is a gram-positive lactic acid bacterium. The scientific classification of L. lactis is: Kingdom: Bacteria; Division: Firmicutes; Class: Bacilli; Order: Lactobacillales; Family: Streptococcaceae Genus: Lactococcus; Species: Lactococcus lactis. The L. lactis may be Lc. cremoris.

In embodiments of the present invention the composition comprises at least two of B. animalis, L. brevis, L. reuteri and L. lactis/Lc. cremoris.

In embodiments of the present invention, the composition may comprise two of bacterial species listed above. For example, in embodiments the composition may comprise B. animalis and L. brevis. In embodiments, the composition may comprise B. animalis and L. reuteri. In embodiments, the composition may comprise B. animalis and L. lactis/Lc. cremoris. In embodiments, the composition may comprise L. brevis and L. reuteri. In embodiments, the composition may comprise L. brevis and L. lactis/Lc. cremoris. In embodiments, the composition may comprise L. reuteri and L. lactis/Lc. cremoris.

In embodiments of the present invention, the composition may comprise three of the bacterial species listed above. For example, in embodiments the composition may comprise B. animalis, L. brevis and L. reuteri. In embodiments, the composition may comprise B. animalis, L. brevis and L. lactis/Lc. cremoris. In embodiments, the composition may comprise B. animalis, L. reuteri and L. lactis/Lc. cremoris. In embodiments, the composition may comprise L. brevis, L. reuteri and L. lactis/Lc. cremoris.

In preferred embodiments of the present invention, the composition may comprise B. animalis, L. brevis, L. reuteri and L. lactis/Lc. cremoris.

In embodiments of the present invention the bacterial species present in the composition may consist of one of the abovementioned combinations, for example B. animalis, L. brevis, L. reuteri and L. lactis/Lc. cremoris.

The present inventors have surprisingly found that these species may be particularly useful in treating dry and/or sensitive skin conditions as they are effective at decreasing the levels of IL-17 and/or IL-23, and/or increasing the level of IL-10 and/or improving the gut barrier. The inventors have further shown that this combination of species results in improved skin health. By utilising this blend of bacterial species the present inventors have developed a composition which can capitalize on the benefits of using blended populations of probiotics that target different physiological aspects of skin disease (e.g. immune aspects and gut-related aspects). Without wishing to be bound by theory, the present inventors hypothesize that by decreasing the levels of these pro-inflammatory interleukins (IL-17 and/or IL-23), and/or increasing the level of the anti-inflammatory interleukin IL-10 and/or improving the gut barrier, an improvement in dry and sensitive skin conditions, particularly psoriasis, can be achieved.

In embodiments in which the composition comprises B. animalis, the B. animalis may comprise, for example, Bifidobacterium animalis subsp. lactis. In embodiments, the B. animalis may comprise, for example, Bifidobacterium animalis subsp. lactis BB-12® and/or strains derived therefrom. In embodiments in which the composition comprises B. animalis, the B. animalis may comprise B. animalis W53 as commercially available from Winclove Probiotics B. V and/or strains derived therefrom.

In embodiments in which the composition comprises L. brevis, the L. brevis may comprise, for example, L. brevis LP9 and/or strains derived therefrom. In embodiments in which the composition comprises L. brevis, the L. brevis may comprise L. brevis W63 as commercially available from Winclove Probiotics B. V and/or strains derived therefrom.

In embodiments in which the composition comprises L. reuteri, the L. reuteri may comprise, for example, L. reuteri FN041 and/or strains derived therefrom. In embodiments in which the composition comprises L. reuteri, the L. reuteri may comprise 30 L. reuteri W192 as commercially available from Winclove Probiotics B. V and/or strains derived therefrom.

In embodiments in which the composition comprises L. lactis, the L. lactis may comprise, for example L. lactis NZ9000 and/or strains derived therefrom. In embodiments in which the composition comprises L. lactis, the L. lactis may comprise Lc. cremoris W224 as commercially available from Winclove Probiotics B. V and/or strains derived therefrom.

The phrase “strains derived therefrom” as used herein, means strains with genomic variations (e.g. insertions, deletions, substitutions) compared to the original strains, but which retain the ability to treat or prevent skin conditions by decreasing the levels of interleukin-17 and/or interleukin-23 in the subject, and/or increasing the levels of interleukin-10 in the subject and/or improving gut barrier function in the subject. The skilled person will be aware of how such strains may be derived, for example through the use of mutagenesis methods well known in the art, for example site-directed mutagenesis or random mutagenesis.

In embodiments of the present invention in which the composition comprises at least two of Bifidobacterium animalis, Lactobacillus brevis, Lactobacillus reuteri and L. lactis/Lc. cremoris, the composition may comprise additional bacterial species, e.g. probiotic bacterial species. Suitable probiotic bacterial species include, for example, bacterial species selected from the group consisting of Lactobacillus sp., Bifidobacterium sp., Lactococcus sp., Streptococcus sp. and combinations thereof. Particularly suitable bacterial species for inclusion in the composition of the present invention include, for example, Lactobacillus acidophilus (for example strain LA5), Lactobacillus casei (for example strain DN001), Lactobacillus plantarum (for example strain HY7714/K8), Lactobacillus pentosus, Lactobacillus paracasei (for example strain CS3), Lactobacillus acidophilus (for example strain IDCC3302), Bifidobacterium infantis (for example strain 35624), Bifidobacterium breve (for example strain B3). Preferably, the additional bacterial species is a bacterial species which decreases the levels of IL-23 and/or IL-17. Preferably, the additional bacterial species is a bacterial species which increases the levels of IL-10. Preferably, the bacterial species is a bacterial species which improves gut barrier function. As shown by the present inventors, bacterial species which exhibit these activities are particularly suitable for the treatment of dry or sensitive skin conditions.

The bacterial species present in the composition of the present invention are live bacterial species i.e. they are living and have not been killed or inactivated. However, it will be appreciated that in addition to containing live probiotic bacteria, the composition of the present invention may further comprise cellular components of said probiotic bacteria (for example, cell wall components, cell membrane components, nucleic acids, proteins etc.), metabolites and/or molecules secreted from said bacteria.

The composition of the present invention may be used in the treatment and/or prevention of a dry and/or sensitive skin condition in a subject.

The term “treatment”, “treat” or “treating” as used herein means that the composition reduces or alleviates signs or symptoms of the dry and/or sensitive skin condition, improves the clinical course of the condition, decreases the number or severity of exacerbations or reduces any other objective or subjective indicia of the condition.

The term “prevention”, “prevent” or “preventing” as used herein means that the composition avoids the occurrence of the condition, particularly when the subject is predisposed to the condition, for example.

The skilled person will appreciate that the phrase “dry and/or sensitive skin condition” encompasses conditions which result in the skin of a subject being itchy, irritable, red, dry, cracked and/or inflamed. Such conditions include, for example, psoriasis, eczema, ichthyosis, dermatitis (e.g. contact dermatitis or atopic dermatitis), acne, rosacea and Netherton's Syndrome.

In embodiments of the invention, the composition is for use in the treatment and/or prevention of psoriasis, atopic dermatitis, contact dermatitis, eczema, ichthyosis, acne, rosacea or Netherton's Syndrome. In preferred embodiments of the invention, the composition is for use in the treatment and/or prevention of psoriasis, atopic dermatitis, contact dermatitis, eczema or ichthyosis.

The use of the composition of the invention advantageously avoids the side effects seen with corticosteroids which are frequently prescribed for the treatment of such conditions.

In preferred embodiments of the invention, the composition is for use in the treatment and/or prevention of psoriasis. As described above, psoriasis can be classified into the following types: plaque, guttate, inverse, pustular and erythrodermic. The composition of the present invention can be used in the treatment and/or prevention of any type of psoriasis.

The present inventors have surprisingly found that the compositions of the present invention may be particularly effective at treating or preventing psoriasis, which is associated with elevated levels of inflammatory markers such as IL-17 and IL-23, an altered microbiome and a leaky gut. The present inventors have shown that subjects suffering from psoriasis saw a marked improvement in skin condition when treated with the probiotic bacterial composition of the present invention. Without wishing to be bound by theory, the inventors hypothesize that the provision of the probiotic bacteria of the invention reduces IL-17 and IL-23 inflammatory markers, and/or increases the level of anti-inflammatory markers, such as IL-10, and/or restores or improves gut function, thereby resulting in improved skin condition.

The term “subject” as used herein, is used to refer to any individual suffering from a dry or sensitive skin condition. In embodiments of the present invention, the subject may be, for example, a human or an animal, for example, a dog, cat, rabbit, bird, cattle, sheep, goat, horse or pony. However, in preferred embodiments, the subject is a mammal, preferably a human.

In the composition of the present invention, the bacterial species present, for example Bifidobacterium animalis, Lactobacillus brevis, Lactobacillus reuteri and/or L. lactis/Lc. cremoris, are present a therapeutically effective amount. The skilled person will appreciate that the term “therapeutically effective amount” is used herein to mean the amount of bacterial species required to produce the effect of treating or preventing a dry and/or sensitive skin condition.

As will be appreciated by the skilled person, the therapeutically effective amount may vary based on characteristics of the subject, for example, age, weight and sex of the subject. The therapeutically effective amount may also vary based on the route of administration.

In embodiments of the present invention, the total concentration of probiotic bacterial species in the composition is between 10⁶ and 10¹² cfu/g. Preferably, the total concentration of probiotic bacterial species in the composition is between 10⁸ and 10¹⁰ cfu/g. More preferably, the total concentration of probiotic bacterial species in the composition is around 10⁹ cfu/g.

In embodiments of the invention, the therapeutically effective amount of bacterial species will be between 10⁶ and 10¹² cfu/day. Preferably, the therapeutically effective amount of bacterial species is between 10⁸ and 10¹⁰ cfu/day. More preferably, the therapeutically effective amount of bacterial species is around 10⁹ cfu/day. When referring here to the therapeutically effective amount of bacterial species, we refer to the total amount of bacterial species being between 10⁶ and 10¹² cfu/day, between 10⁸ and 10¹⁰ cfu/day or around 10⁹ cfu/day.

In embodiments of the present invention, the total concentration of bacterial species Bifidobacterium animalis, Lactobacillus brevis, Lactobacillus reuteri and Lactococcus lactis/Lc. cremoris in the composition is between 10⁶ and 10¹² cfu/g. Preferably, the total concentration of bacterial species Bifidobacterium animalis, Lactobacillus brevis, Lactobacillus reuteri and L. lactis/Lc. cremoris in the composition is between 10⁸ and cfu/g. More preferably, the total concentration of bacterial species Bifidobacterium animalis, Lactobacillus brevis, Lactobacillus reuteri and L. lactis/Lc. cremoris in the composition is around 10⁹ cfu/g.

In embodiments of the invention, the therapeutically effective amount of bacterial species Bifidobacterium animalis, Lactobacillus brevis, Lactobacillus reuteri and Lactococcus lactis/Lactococcus cremoris will be between 10⁶ and 10¹² cfu/day. Preferably, the therapeutically effective amount of bacterial species is between 10⁸ and cfu/day. More preferably, the therapeutically effective amount of bacterial species is around 10⁹ cfu/day. When referring here to the therapeutically effective amount of bacterial species, we refer to the total amount of bacterial species being between 10⁶ and 10¹² cfu/day, between 10⁸ and 10¹⁰ cfu/day or around 10⁹ cfu/day.

The composition of the present invention has been shown to be useful in the treatment or prevention of dry or sensitive skin conditions, for example psoriasis. However, alternatively, or in addition, the composition may be for cosmetic use. The composition may be for cosmetic use in improving the appearance and/or texture of the skin. For example, the composition may result in smoother and/or softer skin, or skin with fewer blemishes, increased firmness, reduced flakiness, reduced redness, reduced skin irritability or itchiness. The present inventors have surprisingly shown that subjects treated with the probiotic composition of the invention often exhibit improved skin health including smoother and softer skin and fewer blemishes.

The skilled person will appreciate that the composition of the present invention could be in any suitable form, for example a solution, gel or powder. In embodiments, the composition is formulated for oral administration to the subject. As will be appreciated by the skilled person, “oral”, as used herein, means administered by mouth or ingested.

Formulations suitable for oral administration include liquids, solutions (e.g., aqueous, non-aqueous), suspensions (e.g., aqueous, non-aqueous), emulsions (e.g., oil-in-water, water-in-oil), elixirs, syrups, electuaries, tablets, granules, powders, capsules, cachets, pills, ampoules and boluses. Alternatively, the composition may be in the form of a food stuff or food additive. The composition may be in the form of a drinkable liquid, a spread and/or powder which can be mixed with a solid or liquid food stuff. The composition could be used as a dietary supplement, for example to be blended with foods/drinks or consumed alongside foods/drinks.

The composition may be encapsulated. Encapsulation techniques will be apparent to the skilled person and the technique employed will be tailored to the required stability of the bacterial species during digestive transit.

The bacterial species may be concentrated and/or freeze dried. Advantageously many probiotic bacteria demonstrate excellent freeze-drying survival.

In embodiments, the composition comprises a powder which can be mixed with a liquid, for example water. The powder may comprise freeze-dried bacterial species.

The composition of the present invention may further comprise one or more pharmaceutically or cosmetically acceptable ingredients or excipients.

Pharmaceutically acceptable ingredients are well known to the skilled person, and include, but are not limited to, pharmaceutically acceptable carriers, adjuvants, excipients, diluents, fillers, buffers, preservatives, antioxidants, lubricants, stabilisers, solubilisers, surfactants (e.g. wetting agents), masking agents, colouring agents, fragrance agents and penetration agents.

The composition may further comprise one or more other active agents, for example, other therapeutic and/or prophylactic agents.

In a further aspect of the present invention there is provided a method for treating and/or preventing a dry and/or sensitive skin condition in a subject, the method comprising administering to the subject a composition comprising at least two bacterial species which decrease the levels of interleukin-17 and/or interleukin-23 in the subject, and/or increase the levels of interleukin-10 in the subject and/or improve gut barrier function in the subject.

Details of the composition are described above in relation to the first and second aspects of the present invention.

In embodiments, the composition comprises at least two of the following bacterial species: Bifidobacterium animalis, Lactobacillus brevis, Lactobacillus reuteri and Lactococcus lactis/Lactococcus cremoris.

The present invention therefore provides a method for treating and/or preventing a dry and/or sensitive skin condition in a subject, the method comprising administering to the subject a composition comprising at least two of the following bacterial species: Bifidobacterium animalis, Lactobacillus brevis, Lactobacillus reuteri and Lactococcus lactis/Lactococcus cremoris.

In embodiments of the method of the present invention, the composition is administered orally to the subject. The present invention therefore provides a method for treating/and or preventing a dry and/or sensitive skin condition in a subject, the method comprising orally administering to the subject a composition comprising at least two of the following bacterial species: Bifidobacterium animalis, Lactobacillus brevis, Lactobacillus reuteri and Lactococcus lactis/Lactococcus cremoris.

As will be appreciated by the skilled person, “oral”, as used herein, means ingested or administered by mouth.

In embodiments of the invention, the composition may be administered to the subject daily for one or more weeks, for example daily for one week, daily for two weeks, daily for three weeks, daily for four weeks, daily for five weeks, daily for six weeks, daily for seven weeks, daily for eight weeks. In embodiments of the invention, the composition may be administered to the subject daily for one or more months, for example daily for one month, two months, three months, four months, five months, six months. In embodiments of the invention, the composition may be administered daily for a prolonged period, for example a year or more. In embodiments of the invention, the composition may be administered to the subject twice or three times daily for one or more weeks.

The composition may be administered to the subject at any time of day. However, preferably the composition is administered on an empty stomach.

The composition may be administered alone or in combination with other treatments, either simultaneously or sequentially.

The present invention also provides the use of a composition in the manufacture of a medicament for the treatment and/or prevention of a dry and/or sensitive skin condition, wherein the composition comprises at least two bacterial species which decrease the levels of interleukin-17 and/or interleukin-23, and/or increase the levels of interleukin—and/or improve gut barrier function.

In embodiments, the composition comprises at least two of the following bacterial species: Bifidobacterium animalis, Lactobacillus brevis, Lactobacillus reuteri and Lactococcus lactis/Lactococcus cremoris.

The present invention therefore provides the use of a composition in the manufacture of a medicament for the treatment and/or prevention of a dry and/or sensitive skin condition, wherein the composition comprises at least two of the following bacterial species: Bifidobacterium animalis, Lactobacillus brevis, Lactobacillus reuteri and Lactococcus lactis/Lactococcus cremoris.

Details of the composition are described above in relation to the first and second aspects of the present invention.

In a further aspect of the invention there is provided a use of a composition in the manufacture of a cosmetic composition for use in improving the appearance and/or texture of the skin, wherein the composition comprises at least two bacterial species which decrease the levels of interleukin-17 and/or interleukin-23, and/or increase the levels of interleukin-10 and/or improve gut barrier function.

There is also provided the use of the composition of the invention for cosmetically improving the appearance and/or texture of the skin. For example, the composition may result in smoother and/or softer skin, or skin with fewer blemishes, increased firmness, reduced flakiness, reduced redness, reduced skin irritability or itchiness.

Details of the composition are described above in relation to the first and second aspects of the present invention.

The described and illustrated embodiments are to be considered as illustrative and not restrictive in character, it being understood that only the preferred embodiments have been shown and described and that all changes and modifications that come within the scope of the inventions as defined in the claims are desired to be protected.

The optional features set out herein may be used either individually or in combination with each other where appropriate and particularly in the combinations as set out in the accompanying claims. The optional features for each aspect or exemplary embodiment of the invention as set out herein are also to be read as applicable to any other aspect or exemplary embodiments of the invention, where appropriate. In other words, the skilled person reading this specification should consider the optional features for each exemplary embodiment of the invention as interchangeable and combinable between different exemplary embodiments.

It should be understood that while the use of words such as “preferable”, “preferably”, “preferred” or “more preferred” in the description suggest that a feature so described may be desirable, it may nevertheless not be necessary and embodiments lacking such a feature may be contemplated as within the scope of the invention as defined in the appended claims. In relation to the claims, it is intended that when words such as “a,” “an,” or “at least one,” are used to preface a feature there is no intention to limit the claim to only one such feature unless specifically stated to the contrary in the claim.

### DETAILED DESCRIPTION OF THE INVENTION

The present invention will now be further described with reference to the following figures which show:

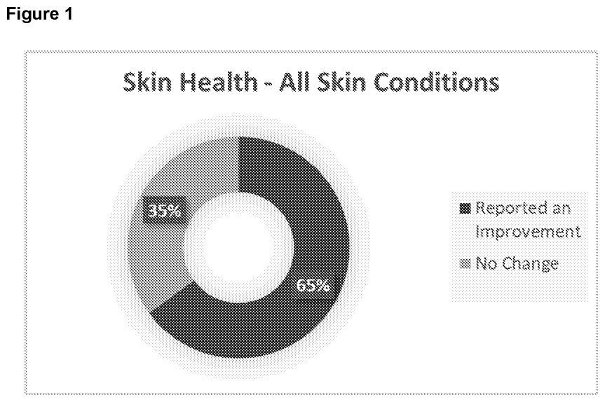

FIG. 1: Skin health improvement in subjects following 56 days treatment with a daily probiotic composition. The percentage of subjects showing an improvement in skin health is shown.

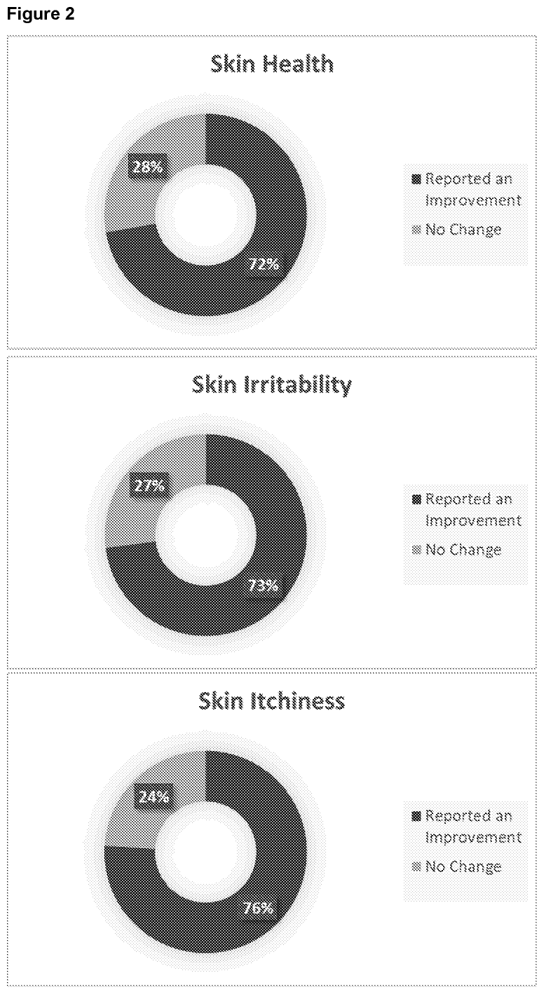

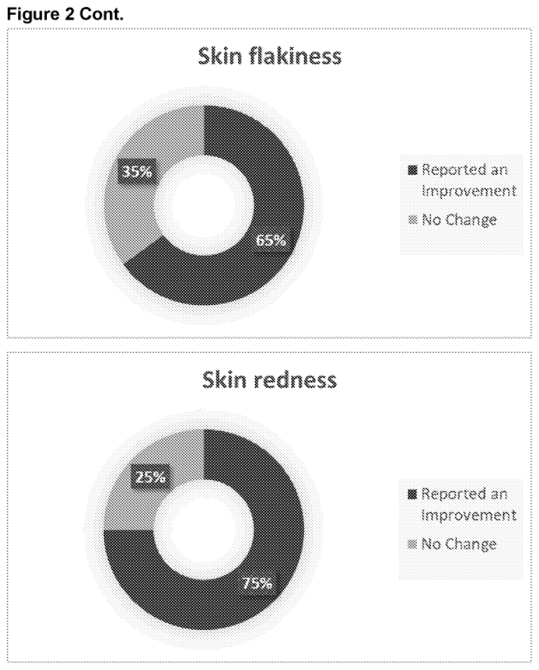

FIG. 2: Skin health outcomes (overall skin health, skin irritability, skin itchiness, skin flakiness and skin redness) in psoriasis subjects. Subjects received a daily probiotic composition for 56 days. The percentage of subjects showing improvement and no change in skin health outcomes is shown.

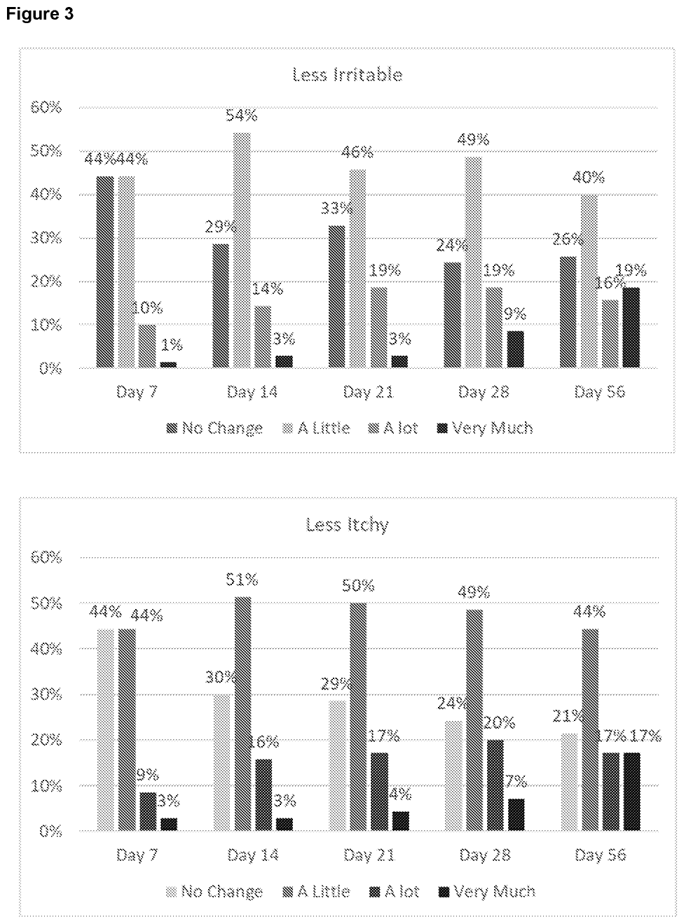

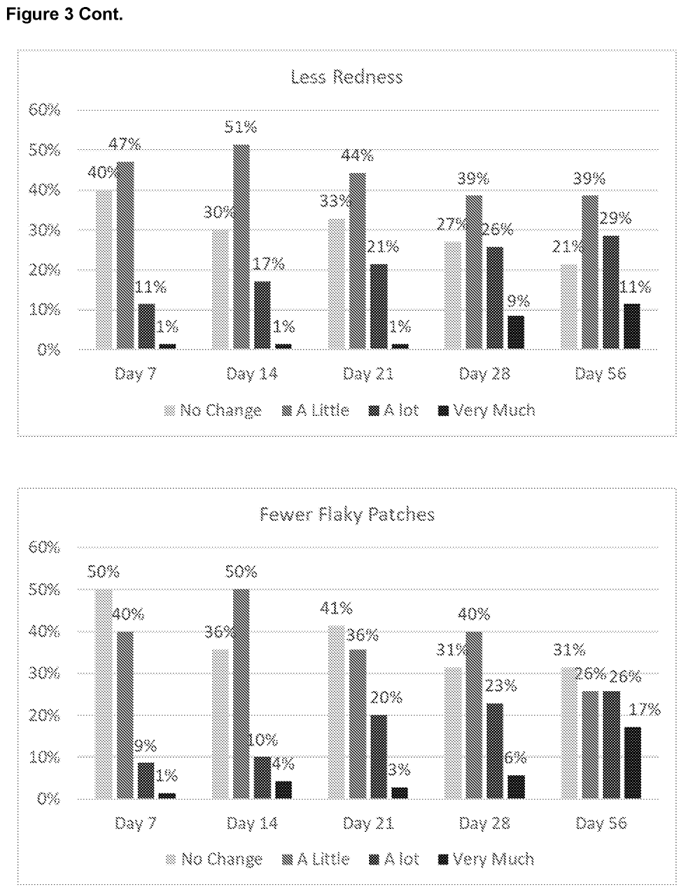

FIG. 3: Skin health outcomes (skin irritability, skin itchiness, skin flakiness and skin redness) over time in psoriasis subjects. The percentage of subjects experiencing no change, a little improvement, a lot of improvement or very much improvement in skin health outcomes over 56 days of treatment with a daily probiotic composition is shown.

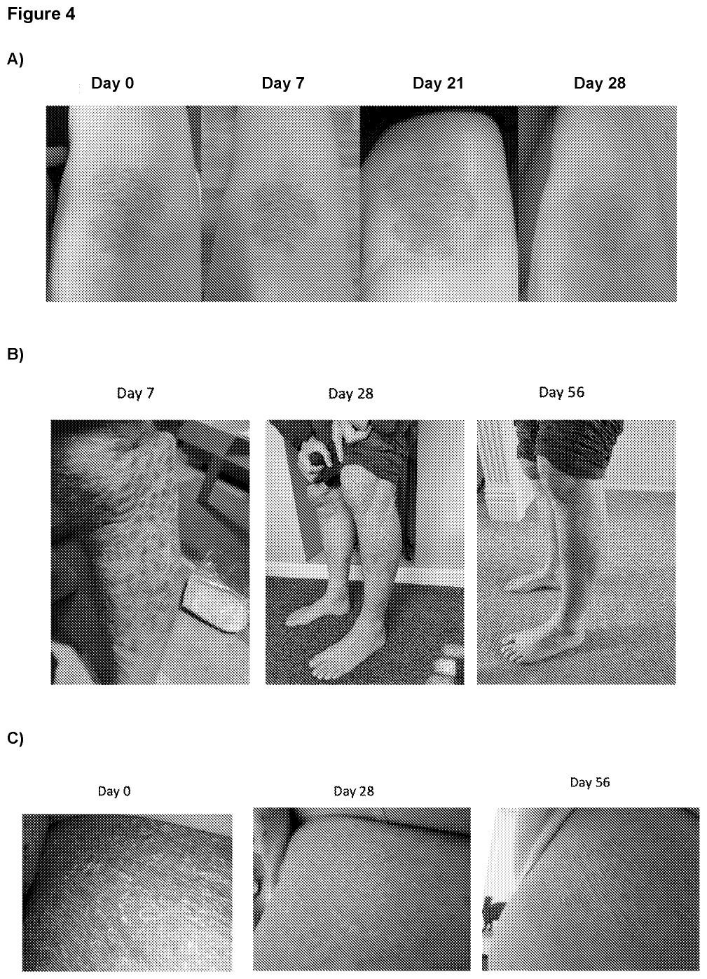

FIG. 4: Images showing skin of psoriasis participants (A, B, C) at various time points throughout the study.

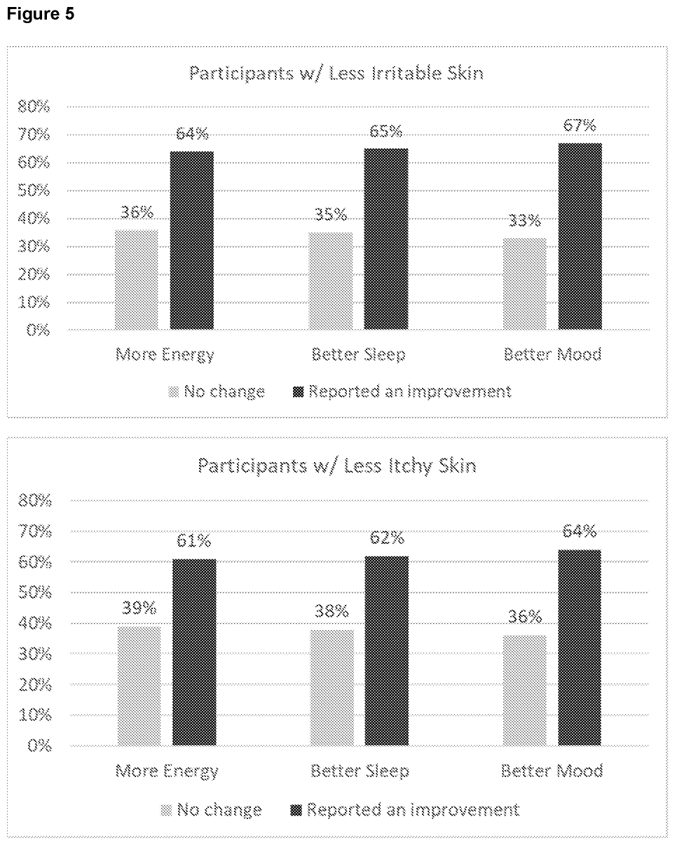

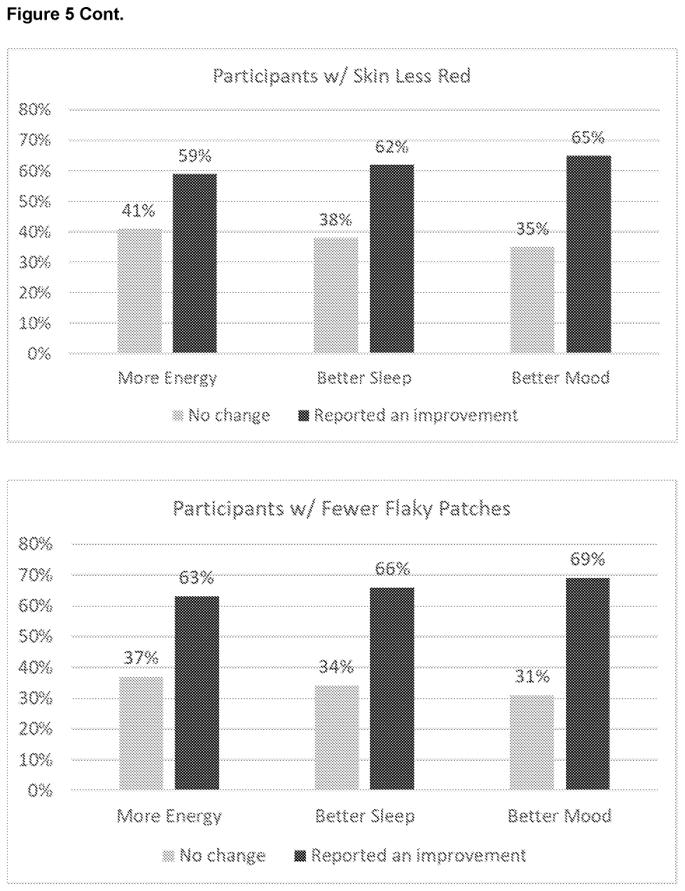

FIG. 5: Energy, sleep and mood benefits in psoriasis participants reporting an improvement in skin health. The percentage of subjects showing improvement or no change in energy, sleep and mood is shown.

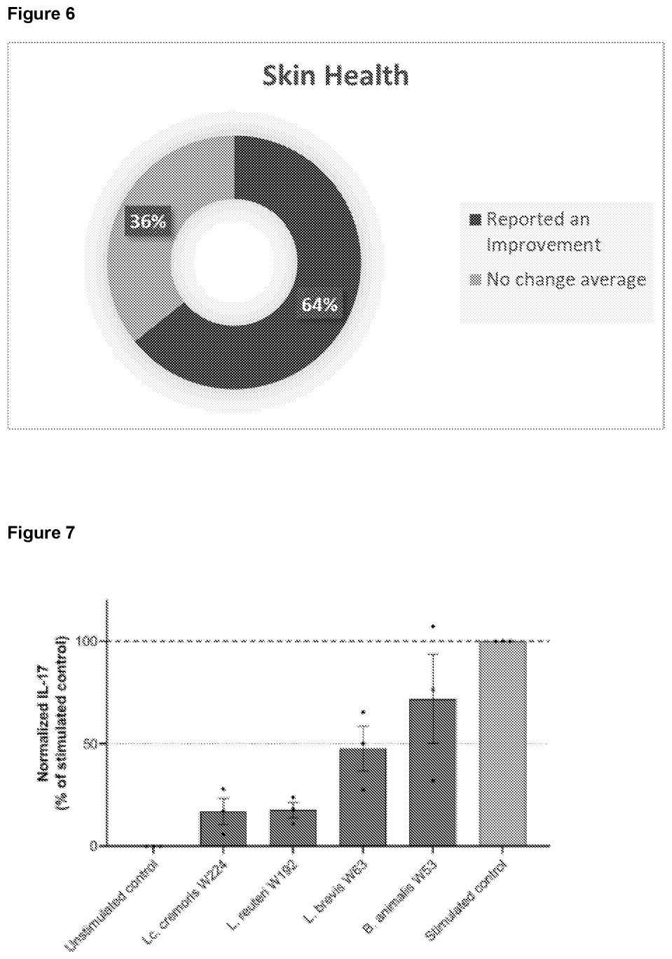

FIG. 6: Skin health improvement in eczema subjects following 56 days treatment with a daily probiotic composition. The percentage of subjects showing an improvement in skin health is shown.

FIG. 7: Effect of Lc. cremoris W224, L. reuteri W192, L. brevis W63 and B. animalis W53 strains on IL-17 produced by anti-CD3/anti-CD28 stimulated PBMCs after 72 hours co-incubation. Unstimulated PBMCs and stimulated PBMCs without the addition of bacterial strains were used as controls. The value of the stimulated control was set at 100% and is indicated by the dotted line. Dots represent individual donor values. Data represent mean values±standard error of the mean.

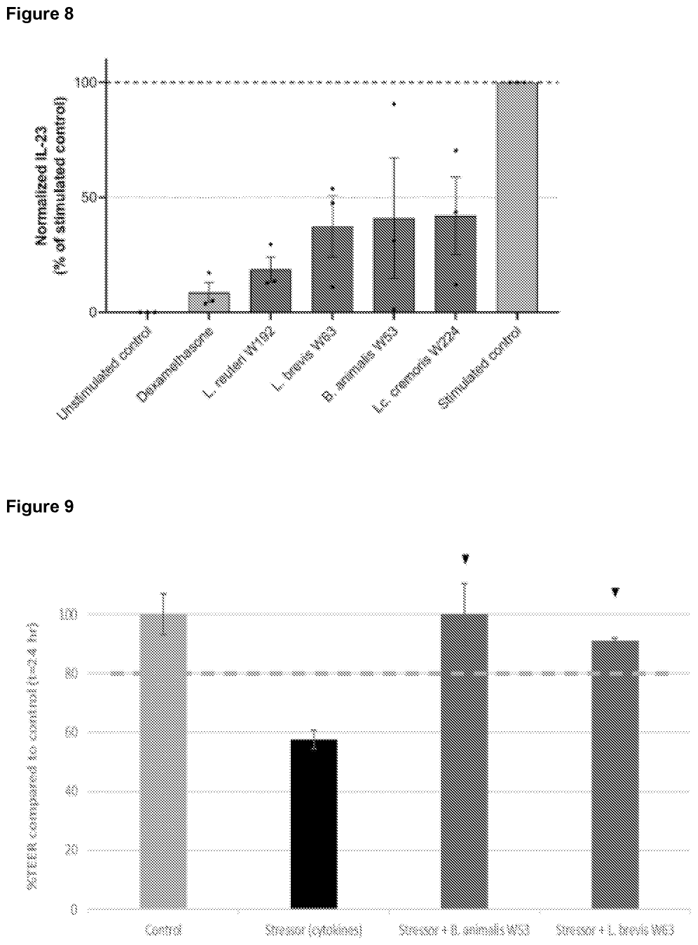

FIG. 8: Effect of Lc. cremoris W224, L. reuteri W192, L. brevis W63 and B. animalis W53 strains on IL-23 produced by activated DCs after 24 hours co-incubation. Unstimulated DCs and DCs stimulated with TH17 cocktail were used as controls. Dexamethasone is used as a positive control for Th17 inhibition. The value of the stimulated control was set at 100% and is indicated by the dotted line. Dots represent individual donor values. Data represent mean values±standard error of the mean.

FIG. 9: Effect of L. brevis W63 and B. animalis W53 on cytokine-induced barrier dysfunction after 24 hours incubation with TNF-α and IL-18. The value of the control, without the addition of any stressor was set at 100%. The dotted line indicates 80% protection of the trans epithelial electric resistance (TEER), and inverted triangles indicates strains with the capacity to protect against cytokine-induced barrier damage. Data represent mean values±standard deviation.

As discussed above, the present invention is based on studies by the inventors in which they have shown that certain probiotic bacterial species and strains are able to reduce IL-17 and IL-23 levels, and/or increase IL-10 levels and/or improve gut barrier function. Since subjects with dry and sensitive skin conditions, such as psoriasis, have been found to have elevated levels of IL-17 and IL-23 and an altered microbiome and leaky gut, the inventors hypothesised that the probiotic bacterial species could be used to treat and prevent dry and sensitive skin conditions. Human consumer studies carried out by the inventors showed that subjects treated with a probiotic bacterial composition according to the invention exhibited improved skin health.

### Example 1— Human Consumer Study

A human consumer study was conducted to establish the benefits of using a composition of the present invention in participants with dry and sensitive skin conditions including psoriasis, atopic dermatitis, contact dermatitis, eczema, acne, rosacea and ichthyosis.

267 participants took part in the study. Participants were male or female and selected based on their history of having a dry or sensitive skin condition (including psoriasis, eczema, rosacea, acne). Participants were excluded if taking antibiotics or if pregnant, breastfeeding or planning on a pregnancy during the study period, or allergic or intolerant to any of the ingredients of the composition.

The participants were provided with a two-month supply of a composition of the invention. Formulation details for the composition used in the study are shown in Table 1 below:

TABLE 1
Formulation of composition used in consumer study

| Ingredient | Per 3 g daily serving |
|---|---|
| Maize starch | |
| Maltodextrin | |
| Manganese sulphate | |
| Potassium chloride | |
| Hydrolysed rice protein | |
| Magnesium sulphate | |
| Bacterial species | |
| Bifidobacterium animalis W53 | ≥3.99 × 10⁹ cfus |
| Lactobacillus brevis W63 | ≥3.99 × 10⁹ cfus |
| Lactobacillus reuteri W192 | ≥3.99 × 10⁹ cfus |
| Lactococcus lactis (Lactococcus | ≥3.99 × 10⁹ cfus |
| cremoris) W224 | |

Before the study, the participants were asked to complete some questions about their lifestyle and skin condition and take a photo of their skin. The study commenced the following day. One sachet (containing 3 g powder) was added to a small glass of lukewarm water, stirred and then drunk. 3 g per day of the composition (one sachet) was taken on an empty stomach. Participants were asked to comment on their skin health every 7 days throughout the study and upload a photo of their skin to assess the effect of the composition on their skin health.

Participants were asked to comment on their general skin health and the following specific skin health outcomes every 7 days during the study: itchiness; irritability; redness; smoothness; softness, blemishes; dryness; flakiness and firmness.

At 56 days following initiation of the study, 65% of participants reported an improvement in skin health (FIG. 1). In particular, 75% reported less itch, 74% reported less irritability, 73% reported less redness, 62% reported less dryness and 64% reported fewer flaky patches. This demonstrates the effectiveness of the composition in treating dry and sensitive skin conditions such as psoriasis, atopic dermatitis, contact dermatitis, eczema, ichthyosis, acne, rosacea or Netherton's Syndrome, for example. The 56-day data also indicated that a significant proportion of participants reported smoother (66%), softer (63%) and firmer (48%) skin with fewer blemishes (59%), indicating that the composition may be effective in promoting cosmetic skin improvement.

Of the participants taking part in the study, 177 had psoriasis. Skin health outcomes specific to psoriasis sufferers include skin irritability, skin itchiness, skin flakiness and skin redness. 56 days after initiation of the study, 91 psoriasis participants reported outcomes. 72% of psoriasis participants reported an improvement in general skin health. Regarding the specific psoriatic outcomes, 73% of psoriasis participants reported an improvement in skin irritability, 76% of psoriasis participants reported an improvement in skin itchiness, 65% of psoriasis participants reported an improvement in skin flakiness and 75% of psoriasis participants reported an improvement in skin redness (FIG. 2). In addition to the psoriasis specific outcomes (irritability, itchiness, flakiness and redness), 69% of the psoriasis participants reported smoother skin, 64% reported softer skin, 62% reported fewer blemishes and 49% reported firmer skin.

Skin health outcomes (itch, irritability, flakiness and redness) in the psoriasis participants were analysed at 7-, 14-, 21-, 28- and 56-days post study initiation with participants indicating whether they experienced no improvement, a little improvement, a lot of improvement or very much improvement (FIG. 3). Interestingly, for all skin health outcomes (itch, irritability, flakiness and redness) the participants reporting a lot and very much improvement increased over time, with between 20 and 50% of psoriasis participants exhibiting a lot or very much improvement in skin health outcomes by 56-days post study initiation. Improvements in skin condition were seen within 7 days and improvements continued for the time of the study.

The participants were also asked to submit photographs of their skin. FIG. 4 shows photographs submitted by a psoriatic participants (A, B, C). As shown in FIG. 4, an improvement in the psoriatic skin condition was evident in the participants, particularly with continued use, the most improvement being seen by day 56. The participants also provided written feedback. Participant B commented “skin is looking better. Less red and not as sore.” And at day 42 “working well and getting better every week”. Participant C commented “big patch on elbow has vanished . . . Also huge solid patch running from back of knee to top of thigh is pretty much flat.”

Psoriasis has a profound psychological and social effect and can induce isolation, and lead to anxiety and depression. As part of the study, participants were asked to comment on various lifestyle outcomes, particularly their energy levels, sleep and mood. From the psoriasis participants that reported a positive change in their skin health (itchiness, irritability, redness and flakiness) on day 56, the majority experienced a benefit in their energy levels, sleep and mood (FIG. 5). For example, of the 73% of participants that reported their skin was less irritable on day 56, 64% felt more energetic, 65% slept better and 67% had a better mood. Similar improvements in well-being were also observed in participants reporting reductions in itch, redness and flaking.

These data demonstrate that participants suffering from psoriasis showed a marked improvement in both skin health and lifestyle outcomes (energy levels, sleep and mood) following oral administration of a probiotic composition including Bifidobacterium animalis, Lactobacillus brevis, Lactobacillus reuteri and Lactococcus lactis (Lactococcus cremoris). These data demonstrate that the composition of the invention is particularly effective at treating dry and sensitive skin conditions such as psoriasis.

35 of the study participants suffered from eczema. At 56 days following initiation of the study, 64% of eczema participants reported an improvement in skin health (FIG. 6). In particular, 71% reported less itch, 71% reported less irritability, 64% reported less redness, 57% reported less dryness and 64% reported fewer flaky patches. In addition, 64% of the eczema participants reported smoother skin, 64% reported softer skin, 64% reported fewer blemishes and 57% reported firmer skin. This data indicates that the composition of the invention is also effective at treating eczema, another dry and sensitive skin condition.

### Example 2— In Vitro Testing

The present inventors performed in vitro testing of a variety of probiotic bacterial species to determine their suitability for use in the treatment of dry or sensitive skin conditions. In particular, the present inventors investigated the effect of these bacterial species on IL-17, IL-23 and IL-10 levels and on gut barrier function (TEER measurement in intestinal epithelial cells) and inflammatory in vitro models (cytokine release by peripheral blood mononuclear cells).

Surprisingly, the present inventors found that certain bacterial species, for example Bifidobacterium animalis, Lactobacillus brevis, Lactobacillus reuteri and Lactococcus lactis were particularly effective at reducing IL-17 and IL-23 levels, and/or increasing IL—levels and/or improving gut barrier function thereby demonstrating their suitability for treating dry and/or sensitive skin conditions such as psoriasis.

### Inhibition of Il-17 Produced by Activated PBMCs

A key role for IL-17 in the pathogenesis of psoriasis has been suggested and therefore reducing IL-17 is considered to be of benefit therapeutically. A major source of IL-17 and Th17 lymphocytes. Therefore, peripheral blood mononuclear cells (PBMCs) were used to investigate the effect of the probiotic bacterial species on IL-17 levels.

### Methods

PBMCs are mononuclear cells isolated from peripheral blood such as lymphocytes, monocytes and macrophages. PMBCs were isolated from three different human donors and stimulated with anti-CD3/anti-CD-28 monoclonal antibodies, an activator of T-lymphocytes, to stimulate an inflammatory state. Subsequently, PBMCs were co-cultured with different probiotic strains. PBMCs cultured with medium only, and PBMCs cultured with medium and stimulant were used as controls for each donor. Freeze-dried probiotic strains were reconstituted in cell culture medium before being added to the PBMC culture. After 72 hours the culture supernatants were collected and IL-17 levels were measured by the multiplex cytokine assay of Luminex.

### Results

The results, shown in FIG. 7, confirm that anti-CD3/anti-CD-28 stimulation increases the secretion of IL-17 by PBMCs in all three donors (right hand bar) compared to the unstimulated control (left hand bar). Following co-culture of bacteria with stimulated PBMCs, the probiotic strains reduce IL-17 secretion. The most pronounced effect was observed with Lc. cremoris (L. lactis) W224 and L. reuteri W192. A beneficial effect was also seen with L. brevis W63 and B. animalis W53.

### Inhibition of Il-23 Produced by Activated Dcs

A central role of the IL-23/IL-17 axis in the immunopathogenesis of psoriasis has been proposed. IL-23, produced by activated dendritic cells (DCs), is central to the survival and proliferation of Th17 lymphocytes and thus stimulates IL-17 production by these cells. This increase of cytokines such as IL-17 results in an inflammatory cascade leading to psoriatic disease. Hence, reducing IL-23 is considered to be of therapeutic value to psoriasis patients to reduce inflammation. Therefore, the effect of probiotic bacterial strains on IL-23 production in vitro by DCs was investigated.

### Methods

PBMCs were isolated from three different human donors. CD14+ monocytes were subsequently purified and differentiated to Th17 DCs for 7 days. Subsequently, DCs were co-cultured with different probiotic strains. Untreated and dexamethasone treated DCs were used as controls for each donor. Dexamethasone is used as a positive control for Th17 inhibition. Freeze-dried probiotic strains were reconstituted in cell culture medium before being added to the DC culture. After 6 hours incubation with the probiotic strain, a TH17 cocktail was added to the DCs. Supernatant was harvested after 24 hours co-culture with probiotic strains. IL-23 was measured by Luminex in duplicates.

### Results

The results, shown in FIG. 8, confirm that the TH17 cocktail induces IL-23 secretion in all three DC donors compared to the unstimulated control. Dexamethasone, a positive control for Th17 inhibition, inhibits the secretion of IL-23 convincingly in all three donors (FIG. 8). Following co-culture of the bacterial strains with stimulated DCs, L. reuteri W192, L. brevis W63, B. animalis W53 and Lc. cremoris (L. lactis) W224 reduced IL-23 secretion. L. reuteri W192 and L. brevis W63 reduced IL-23 secretion to 55% compared to stimulated DCs in all three donors.

### Inhibition of Cytokine-Induced Barrier Degradation (TEER)

The intestinal barrier plays an essential role in the communication between the gut and the rest of the human body. Maintenance of the intestinal epithelial integrity and thus barrier function is critical for essential physiological processes and increased permeability can lead to increased levels of bacterial antigens in the circulating blood which can lead to systemic low-grade inflammation, a hallmark of various diseases such as psoriasis. The inventors therefore sought to measure the effects of probiotic bacterial species of the intestinal epithelial barrier. Measuring trans epithelial electrical resistance (TEER) is a widely accepted quantitative technique to measure the integrity of tight junction dynamics in cell culture models of epithelial monolayers. A reduction in TEER indicates that the intestinal barrier function is impaired.

### Methods

A monolayer of Caco-2 cells was first exposed to the probiotic bacterial strains for 2 hours followed by exposure to an inflammatory stressor (TNF-α and IL-18) in the presence of the same probiotic bacterial strain. After 24 hours the TEER of the monolayer was measured. The results were compared to the TEER of a Caco-2 monolayer that was exposed to stressor alone and a Caco-2 monolayer that was not exposed to the stressor. This last control was set of 100% (see FIG. 9). Each condition was measured in duplicate.

### Results

As shown in FIG. 9, B. animalis W53 and L. brevis W63 showed a pronounced effect on protecting the epithelial cells against cytokine-induced barrier dysfunction. They were able to maintain >90% of the TEER following inflammatory stress, and thus showed a beneficial effect on gut barrier function.

### Example 3— Human Clinical Study

In order to further develop the composition of the present invention, the present inventor planned a human clinical study to take place at a dermatology unit in North-West England.

Participants were male or female, aged 18-70 and selected based on their history of having stable plaque psoriasis with PASI score higher than 3 and no flare-up in the four weeks prior to the beginning of the study. Participants were excluded if taking antibiotics, if suffering from a GI disease or on a special diet, having had phototherapy or systemic therapy (with the exception of methotrexate) in the last three months, having taken a probiotic and/or prebiotic in the two weeks preceding the trial, if pregnant, breastfeeding or planning on a pregnancy during the study period, or allergic or intolerant to any of the ingredients of the composition.

The study products will be blinded to the participants and clinical team. Each participant will be asked to take the study product daily for a total of 6 months. The study has two arms, each of which will recruit 50 volunteers with mild to moderate psoriasis (25 placebo, 25 inventive composition). Arm one: psoriasis sufferers who are currently prescribed methotrexate; and arm two: psoriasis sufferers who are treatment naive and not prescribed medication. Each participant will visit the centre for assessment on week 1, week 6, week 12 and week 26. Skin, blood and stool samples will be taken at each visit for microbiome analysis and biomarkers (IL1β, IL17, IL23, and TNF-α) and disease severity assessments will be performed. Participants will complete a weekly questionnaire.

### Outcomes Will be Assessed at the End of the Study

It will be appreciated that numerous modifications to the above-described method may be made without departing from the scope of the invention as defined in the appended claims. For example, although a powdered form of the composition is exemplified, it will be appreciated that the composition may be in any suitable form, for example a liquid, solution, suspension, syrup, tablet, granules or capsule. Furthermore, although the examples focus on composition comprising Bifidobacterium animalis, Lactobacillus brevis, Lactobacillus reuteri and Lactococcus lactis (Lactococcus cremoris), other suitable probiotic bacteria may be included in the composition of the present invention.

1. A method for treating or preventing a dry and/or sensitive skin condition in a subject, comprising administering a composition comprising at least two bacterial species which decrease the levels of interleukin-17 and/or interleukin-23 in the subject, increase the levels of interleukin-10 in the subject and/or improve gut barrier function in the subject.

2. The method according to claim 1, wherein the bacterial species are selected from: Bifidobacterium animalis, Lactobacillus brevis, Lactobacillus reuteri, Lactococcus lactis/Lactococcus cremoris, Lactobacillus acidophilus, Lactobacillus casei, Bifidobacterium infantis, Lactobacillus plantarum, Lactobacillus pentosus, Lactobacillus paracasei, Lactobacillus acidophilus and Bifidobacterium breve.

3. The method according to claim 1, wherein the composition comprises at least two bacterial species selected from the group consisting of: Bifidobacterium animalis, Lactobacillus brevis, Lactobacillus reuteri and Lactococcus lactis/Lactococcus cremoris.

4. The method according to claim 1, wherein the composition comprises at least three bacterial species selected from the group consisting of: Bifidobacterium animalis, Lactobacillus brevis, Lactobacillus reuteri and Lactococcus lactis/Lactococcus cremoris.

5. The method according to claim 1, wherein the composition comprises B. animalis, L. brevis, L. reuteri and L. lactis/Lc. cremoris.

6. The method according to claim 1, wherein the dry and/or sensitive skin condition comprises psoriasis, atopic dermatitis, contact dermatitis, eczema or ichthyosis.

7. The method according to claim 6, wherein the dry and/or sensitive skin condition comprises psoriasis.

8. The method according to claim 1, wherein the bacterial species are present in the composition at a concentration of between 10⁶ and 10¹² cfu/g.

9. The method according to claim 1, wherein the composition is formulated for oral administration.

10. The composition for use method according claim 1, wherein the composition comprises a powder.

11. A composition comprising at least two bacterial species which decrease the levels of interleukin-17 and/or interleukin-23, increase the levels of interleukin-10 and/or improve gut barrier function.

12. The composition according to claim 11, wherein the composition comprises at least two bacterial species selected from the group consisting of:
Bifidobacterium animalis, Lactobacillus brevis, Lactobacillus reuteri and Lactococcus lactis/Lactococcus cremoris.

13. The composition according to claim 11, wherein the composition comprises B. animalis, L. brevis, L. reuteri and L. lactis/Lc. cremoris.

14. The composition according claim 11, wherein the bacterial species are present in the composition at a concentration of between 10⁶ and 10¹² cfu/g.

15. The composition according claim 11, wherein the composition is formulated for oral administration.

16. The composition according claim 11, wherein the composition comprises a powder.

17. The composition according to claim 14, wherein the bacterial species are present in the composition at a concentration of about 10⁹ cfu/g.

18. The method according to claim 8, wherein the bacterial species are present in the composition at a concentration of about 10⁹ cfu/g.
